(19) United States
(12) Patent Application Publication
(10) Pub. No.: US 2005/0059101 A1
(43) Pub. Date: Mar. 17, 2005

(54) Bivalent targeting of cell surfaces

(76) Inventor: Gordon Ringold, Los Altos Hills, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/938,863

(22) Filed: Sep. 10, 2004

Related U.S. Application Data
(60) Provisional application No. 60/501,678, filed on Sep. 10, 2003.

(51) Int. Cl. G01N 33/574; A61K 39/395; C07K 16/30

(52) U.S. Cl. 435/7.23; 530/388.8; 530/391.1

(57) ABSTRACT

Bivalent molecules that target cancer cells by simultaneously binding to at least two different compounds, and methods for their use in treating, diagnosing, imaging and staging cancers are provided.

This application claims the benefit under 35 U.S.C. § 119 of U.S. Provisional Patent Application Ser. No. 60/501,678, filed Sep. 10, 2003, entitled “Bivalent Targeting Of Cell-Surface Antigens,” and incorporated by reference herein in its entirety.

### FIELD OF THE INVENTION

The present invention is directed to methods for treating, diagnosing, imaging and staging cancers. Specifically, the present invention is directed to the use of bivalent molecules that target cancer cells by simultaneously binding to at least two different compounds.

### BACKGROUND OF INVENTION

Cancer therapeutics have been conjugated to antibodies to obtain selective cytotoxicity. Such target-directed therapies have a number of advantages over non-targeted therapies. Most significantly, target-directed therapies allow doses of a therapeutic agent to be delivered directly to the site of action while largely sparing normal, healthy tissue from the toxic effects.

Target-directed therapies have shown efficacy in treating hematological tumors. Two radiolabeled monoclonal antibodies—Zevalin® (ibritumomab tiuxetan) and Bexxar® (tositumomab)—have been approved for use in treating certain refractory forms of non-Hodgkin's lymphoma. Both Zevalin and Bexxar target the CD20 antigen expressed on the surface of malignant and normal B-lymphocytes.

Solid tumors have also been successfully treated using targeted therapies. In 1998, the FDA approved a monoclonal antibody—Herceptin® (trastuzumab)—to treat certain refractory forms of breast cancer. Human epidermal growth factor receptor 2 (HER2), found on the surface of some normal cells and important in cell growth regulation, is over-expressed in some breast cancers. Herceptin acts by binding to HER2, thereby blocking the receptor.

The use of radiolabeled antibodies to treat solid tumors also has been investigated. The size of antibody conjugates limits penetration of the therapeutic agent into solid tumors. In several studies, the radioactive dose delivered to the tumor was ultimately deemed inadequate. Furthermore, host toxicity remains a problem. An antigen used to target cancer cells may also be present in normal, healthy tissues. In addition, antibody conjugates are not rapidly eliminated and can damage sensitive tissues, such as bone marrow, before they are cleared from circulation. Prior art treatment methods have not provided complete solutions to these problems.

Radioisotope-linked antibodies also have applications in cancer diagnostics. Prostascint® (capromab pendetide), for example, is in current use as a diagnostic imaging agent for prostate cancer. The Prostascint antibody targets prostate specific membrane antigen (PSMA). The antibody is labeled with In-111 which can be directly imaged after uptake by the cancer. A variety of radioactive isotopes have been attached to antibodies against tumor cell surface antigens. However, clearance and specificity issues limit broader application of this approach.

Thus, there is a need to specifically deliver therapeutic agents to a tumor while sparing the toxic side effects of the therapeutic to normal tissue.

### SUMMARY OF THE INVENTION

This invention provides a bivalent molecule that comprises a therapeutic agent and two binding moieties, each binding moiety being specific for a different compound produced by or associated with cancer cells (e.g., a cell-surface antigen). This invention also provides a method for treating cancer by administering a therapeutically effective amount of the bivalent molecule to a patient.

This invention also provides a bivalent molecule that comprises an imaging agent and two binding moieties, each binding moiety being specific for a different compound produced by or associated with cancer cells (e.g., a cell-surface antigen). This invention also provides a method for diagnosing, staging and imaging cancer by administering the bivalent molecule to a patient and subsequently measuring the location, extent or distribution of the imaging agent.

The invention also provides a pharmaceutical composition comprising a therapeutically effective amount of the bivalent molecule of the invention in a pharmaceutically acceptable adjuvant.

### DETAILED DESCRIPTION OF THE INVENTION

The bivalent molecule of the present invention comprises a therapeutic agent and two binding moieties, each binding moiety specific for a different molecule produced by or associated with a cancer cell. In some embodiments, the binding moiety is specific for the extra-cellular domain of a cell-surface molecule. The molecule may be associated with the cell by covalent or non-covalent means (e.g., a secreted protein at the cell surface). Having two binding moieties, each specific for a different molecule, increases the bivalent molecule's specificity for cancer cells expressing both molecules. Although some normal cells may express one of the targeted molecules, very few (if any) may express both.

The bivalent molecule of the invention is capable of binding to two different molecules at the same time, thereby forming a molecular complex. The bivalent molecule is not limited to having two and only two binding moieties; it can be a polyvalent molecule (e.g., trivalent, tetravalent, pentavalent, and so on) or a molecule comprised of linked monovalent molecules so long as at least two binding moieties of the type described above are provided.

In some embodiments, the bivalent molecule is or comprises a polypeptide. In such embodiments, the bivalent molecule may be entirely composed of polypeptide sequences or contain one or more non-peptidic substituents in addition to polypeptide sequences. In other embodiments, the bivalent molecule can be entirely non-protein (e.g., an oligonucleotide), provided that it can form a sufficiently stable complex with the targeted molecules and effectively deliver the therapeutic agent to the tumor.

In some embodiments, the bivalent molecules of the invention are resistant to proteolytic or other cleavage.

The therapeutic agent of the bivalent molecule may be a radioisotope, a drug or a toxin. In some embodiments, the therapeutic agent is a radioisotope. Appropriate radioisotopes that may be used in connection with the present invention include 1-131, 1-125, At-21 1, P-32, P-33, Sc-47, Cu-64, Cu-67, As-77, Y-90, Ph-105, Pd-109, Ag-111, Pr-143, Sm-153, Th-161, Ho-166, Lu-177, Re-186, Re-188, Re-189, Ir-194, Au-199, Pb-212, Bi-212, or any other radioisotope useful for killing tumor cells. For a number of therapeutic embodiments, I-131, Y-90, Cu-67, 1-125, and Bi-212 are particularly useful. Alternatively, in certain embodiments where the treatment method includes subsequent external irradiation of the tumor, the therapeutic agent may be a boron addend.

The effectiveness of a given radioisotope in killing cancer cells is related to the size of the tumor and the individual energy emissions of the radioisotope. Different radioisotopes are known in the art to be effective over different distances. For treating larger tumors, high-energy radioisotopes are useful as therapeutic agents because they are highly penetrating. For treating smaller tumors or individual cells, low or medium energy radioisotopes are useful because they exert their effects over much shorter distances.

In addition to radioisotopes, many drugs and toxins are known to have cytotoxic effects on cancer cells and can be used in connection with the present invention. Examples of known cytotoxic agents useful in the present invention include taxol, nitrogen mustards, alkyl sulfonates, nitrosoureas, and folic acid analogs.

Any binding moiety that is capable of specifically binding to a compound produced by or associated with cancer cells (e.g., cell-surface markers) is included within the invention. The binding moieties are, in some, embodiments, non-antibody species such as proteins, peptides, polypeptides, glycoproteins, lipoproteins, phospholipids, antibody fragments, cell or tissue specific peptides or enzymes. The binding moieties may also be oligonucleotides, steroids, alkaloids, hormones and other receptor-binding molecules.

The binding moieties may recognize any portion of the targeted molecules. In some embodiments, a binding moiety has a specificity for the molecule of at least 65%, at least 60%, or at least 55% and a cross-reactivity to other molecules of less than 30%, less than 35%, less than 40%. In some embodiments, the binding moieties are small in size to maximize the ability of the bivalent molecule to penetrate more deeply into tumor tissue (which is often poorly vascularized). Smaller molecules are also more rapidly cleared from circulation. The binding moieties can be linked in any of a number of ways including without limitation, disulfide bonds, peptide bridging, amide bonds, and other natural or synthetic linkages known in the art.

Binding moieties selectively recognizing the cancer-associated molecules can be identified by a number of methods using techniques known in the art. For example, a population of candidate molecules first may be screened for an ability to compete with an antibody (or other molecule) known to bind to the targeted molecule. Then, a suitable assay may be performed to determine the candidate molecule's ability to bind to the target molecule.

Alternatively, peptides corresponding to the targeted molecule may be synthesized for use as a binding partner in binding assays with random peptide libraries prepared by techniques known in the art, such as, for example, peptide chemistry or phage display. In phage display, for example, bacteriophage displaying random peptide sequences on the phage surface can be allowed to bind to immobilized synthetic target molecule and then the peptide sequence specific for the target molecule can be determined. In addition, stringency washes can be used to isolate those peptide sequences of moderate affinity. Identified peptides can then be synthesized in a bivalent form and tested for the ability simultaneously to bind to different target molecules by any of the methods known in the art.

In other embodiments, peptides can be designed based on an analysis of the amino acid sequence of the target molecule. A bivalent molecule then can be synthesized containing two or more of such designed peptides linked or fused to each other and possibly to an additional polypeptide structure provided as a scaffold for stability or some other purpose (e.g., attachment of the therapeutic agent).

A binding moiety may also be a nonprotein species identified and produced by techniques known in the art, such as, for example, peptidomimetics. Such “mimetics” can be produced by rational drug design based on molecular modeling and the polypeptide sequence of the targeted molecule. Alternatively, combinatorial chemical libraries can be screened (e.g., competitive inhibition of a known antibody binding weakly to the molecule) to identify a compound that selectively binds to an target molecule and then the compound can be synthesized in a bivalent form and tested for the ability simultaneously to form a complex with two different target molecules by any of the methods known in the art.

The binding moiety may be an antibody fragment. As used herein, the term “antibody” refers to polyclonal antibodies, monoclonal antibodies, humanized antibodies, single-chain antibodies, and fragments thereof such as F_{ab}, F(ab′)2, Fv, and other antibody fragments which retain the antigen binding function of the parent antibody. A monoclonal antibody refers to an antibody of uniform light and heavy chain composition that may be produced by a single hybridoma, hybrid hybridoma or trioma clone or by recombinant technology. The term monoclonal antibody is not limited to a particular species or source of the antibody, nor is it intended to be limited by the manner in which it is made. Rather, monoclonal antibody encompasses whole immunoglobulins as well as fragments such as Fab, F(ab′)2, Fv, and other antibody fragments that retain the antigen binding function of the parent monoclonal antibody. Recombinant forms of these antibodies or fragments may be produced in any expression system conventional in the art, such as prokaryotic, as in E. coli, or eukaryotic, as in yeast, insect or mammalian cells. Methods of antibody production and isolation are well known in the art. See, for example, Harlow and Lane (1988) Antibodies: A Laboratory Manual, Cold Spring Harbor Laboratory, New York.

Monoclonal antibodies of any mammalian species can be used in this invention, including but not limited to human, mice, rats, rabbits, goats, sheep, bovine, porcine and equine or combinations thereof. Antibodies of murine or rat origin are preferred in view of the availability of murine or rat cell lines for use in making the required hybrid cell lines and hybridomas to produce the monoclonal antibodies.

As used herein, the term “humanized antibody” means that at least a portion of the framework regions of an immunoglobulin is derived from human immunoglobulin sequences.

As used herein, the term “single chain antibody” refers to an antibody prepared by combining the binding domains (both heavy and light chains) of an antibody with a linking moiety that preserves the binding function. This forms, in essence, a radically abbreviated antibody, having only that part of the variable domain necessary for binding to the antigen. Methods for preparing single chain antibodies are known in the art. Single chain antibodies also include single chain V region fragments (“scFv”) of the antibodies contemplated herein. Single chain V region fragments are made by linking L and/or H chain V regions by using a short linking peptide. Methods for preparing scFv's, Fab libraries, and other antibody referred to herein are known in the art. See, e.g., Antibody Phage Display Methods and Protocols December 2001, Methods in Molecular Biology: Volume #: 178, Humana Press. See also, Diagnostic and Therapeutic Antibodies, August 2000; Methods in Molecular Medicine, Volume #: 40, Humana Press.

Bivalent molecules of the present invention may be a molecule that comprises any antibody that has binding specificity for two different antigens, whether naturally occurring or synthetically made in vitro. This includes molecules formed by chemically conjugating two different antibodies, or produced from a “hybrid hybridoma,” a cell fusion of two monoclonal antibody-producing cells. Methods for preparing such antibodies are known to those skilled in the art.

Antibody-engineering technology allows development of smaller fragments, such as single chain Fv molecules, that are able to specifically bind the antigen without excessive cross-reactivity. Other antibody fragments that may be useful in the present invention include F(ab′)₂, F(ab)₂, Fab′, Fab, Fv and any other fragments of comparable or smaller size retaining the antigen-binding site (including hybrid fragments and genetically engineered and/or recombinant antibodies and proteins). Mixtures of antibody fragments may be used according to the present invention. For example, the bivalent molecule may be composed of two different monospecific antibody fragments that specifically bind to an antigen, thus creating a bivalent molecule with dual specificity.

The binding moiety can also be an antibody, such as an IgA, IgE, IgM, IgG or IgD antibody. Antibodies of the present invention can be either polyclonal or monoclonal antibodies. Antibodies useful in the present invention include functional equivalents such as antibody fragments and genetically-engineered antibodies, including single chain antibodies, that are capable of selectively binding to at least one of the epitopes of the protein used to obtain the antibodies. Antibodies of the present invention also include chimeric antibodies that can bind to more than one epitope.

However, as discussed above, the size of the antibody may limit penetration of the therapeutic agent into solid tumors. Furthermore, the bivalent molecules comprising antibodies as the binding moieties may be more slowly eliminated and thus more likely to damage sensitive tissues, such as bone marrow, before they are cleared from circulation. If antibodies are used, they should be carefully selected, based upon factors such as size of the antibody and resistance to enzymatic cleavage. Methods of generating antibodies that are specific for antigens (e.g., monoclonal antibodies) are well-known in the art.

The binding moieties contemplated include polypeptide fragments of the antibodies described elsewhere herein. The size of the polypeptides can be only the minimum size required to provide the desired binding function. Typically, the polypeptides will comprise CDR amino acid sequences. The polypeptides can optionally comprise additional sequence, either native to the antibody, or from a heterologous source, as desired.

The invention includes polypeptides which are functionally equivalent to the antibody from which it is derived, or have altered but measurable binding activity. Modified polypeptides with improved activity are also contemplated. Examples of modified polypeptides include those with conservative substitutions of amino acid residues, and one or more deletions or additions of amino acids which do not significantly deleteriously alter the immunologic activity.

The polypeptides of this invention can be made by any suitable procedure, including proteolysis of the antibody, by recombinant methods or by chemical synthesis. These methods are known in the art and need not be described in detail herein. Examples of proteolytic enzymes include, but are not limited to, trypsin, chymotrypsin, pepsin, papain, V8 protease, subtilisin, plasmin, and thrombin. Intact antibody can be incubated with one or more proteinases simultaneously or sequentially. Alternatively, or in addition, intact antibody can be treated with disulfide reducing agents. Peptides can then be separated from each other by techniques known in the art including, but not limited to, gel filtration chromatography, gel electrophoresis, and reverse-phase HPLC.

The binding polypeptides can also be made by expression from a polynucleotide encoding the peptide according to the information provided elsewhere in this application, in a suitable expression system. Typically, polynucleotides encoding a polypeptide are ligated into an expression vector under control of a suitable promoter and used to genetically alter the intended host cell. Both eukaryotic and prokaryotic host systems can be used. The polypeptide is then isolated from lysed cells or from the culture medium and purified to the extent needed for its intended use. Examples of prokaryotic host cells appropriate for use with this invention include E. coli. Examples of eukaryotic host cells include avian, insect, plant, and animal cells including, but not limited to, COS7, HeLa, and CHO cells.

Methods of preparing bivalent molecules are known in the art. For example, two binding moieties having different specificities may be linked using a chemical crosslinking agent such as SPDP (N-succinimidyl 3-(2-pyridyldithio)-propionate), o-phenylenedimaleimide (o-PDM), or other crosslinking agents conventional in the art. Other known methods include heterodimerization of Escherichia coli-expressed Fab fragments through cysteine residues or leucine zippers. Protein engineering has permitted the design of even smaller bispecific fragments based on single-chain Fv fragments (scFv) fragments, which may be formed in vivo by noncovalent association of two single-chain fusion products. The bivalent molecule may be prepared by linking the two binding moieties, each optionally conjugated to a spacer group, via their respective chemical functionalities via the linkages shown in Table 1. Those of skill in the art will recognize that one can first attach the spacer to either binding moiety.

TABLE 1

| Chemical | Chemical | |
|---|---|---|
| Functionality 1 | Functionality 2 | Linkage |
| Hydroxy | Carboxy | Ester |
| | Hydroxy | Carbonate |
| | Amine | Carbamate |
| | SO₃ | Sulfate |
| | | Phosphate |
| | Carboxy | Acyloxyalkyl |
| | | ether |
| | Ketone | Ketal |
| | Aldehyde | Acetal |
| | Hydroxy | Anhydride |
| Mercapto | Mercapto | Disulfide |
| | Carboxy | Acyloxyalkyl |
| | | thioether |
| | Carboxy | Thioester |
| Carboxy | Amino | Amide |
| | Mercapto | Thioester |
| | Carboxy | Acyloxyalkyl |
| | | ester |
| | Carboxy | Acyloxyalkyl |
| | | amide |
| | Amino | Acyloxyalkoxy |
| | | carbonyl |
| | Carboxy | Anhydride |
| | Carboxy | N-acylamide |
| | Hydroxy | Ester |
| | Hydroxy | Hydroxymethyl |
| | | ketone ester |
| | Hydroxy | Alkoxycarbonyl |
| | | oxyalkyl |
| Amino | Carboxy | Acyloxyalkyl |
| | | amine |
| | Carboxy | Acyloxyalkyl |
| | | amide |
| | Amino | Urea |
| | Carboxy | Amide |
| | Carboxy | Acyloxyalkoxy |
| | | carbonyl |
| | Amide | N-Mannich base |
| | Carboxy | Acyloxyalkyl |
| | | carbamate |
| Phosphate oxygen | Hydroxy | Phosphate ester |
| | Amine | Phosphoramidate |
| | Mercapto | Thiophosphate |
| | | ester |
| Ketone | Carboxy | Enol ester |
| Sulfonamide | Carboxy | Acyloxyalkyl |
| | | sulfonamide |
| | Ester | N-sulfonyl- |
| | | imidate |

One skilled in the art will readily appreciate that many of these linkages may be produced in a variety of ways and using a variety of conditions.

Alternatively, the two different binding specificities can be combined in a single molecule using a linker. An Fc portion of an antibody is one example of a linker. Other examples of a suitable linker is a helical peptide linker, such as that described by Newton C R, et al., Protein Expression and Purification, 1994 5(5):449-57); the “tag” peptide described by Tu, et al., Journal of Biological Chemistry, 1995 270(16):9322-6); the hinge-like region of B7-1 or B7-2; a peptide segment or a second functional domain such as an Ig; a growth hormone; an adhesion receptor; the FLAG peptide sequence described by Knappik, et al., Biotechniques, 1994 (4):754-61); the Flag peptide consisting of the 11-amino-acid leader peptide of the gene product from bacteriophage T7, as described by Witzgall, et al., Analytical Biochemistry, 1994 223(2):291-8); the “Strep tag” decribed by Schmidt et al. Journal of Chromatography A, 1994 676(2):337-45); the influenza virus hemagglutinin (HA) epitope tag as described by Chen et al., PNAS, Jul. 15, 1993 90(14): 6508-12); the 14-amino acid oligopeptide in simian virus 5 (SV5) described by Hanke, et al., Journal of General Virology, 1992, 73:653-60; the tetrapeptide described by Studer, et al., Bioconjugate Chemistry, 1992 3(5):424-9); the myc epitope as described in Simons, et al, Intracellular routing of human amyloid protein precursor: axonal delivery transport to the dendrites. Journal of Neuroscience Research, 1995 41(1):121-8); the seven-histidine tag of Parks, et al., 1995 210(1):194-201 and Vouret-Craviari, et al., Journal of Biological Chemistry, 1995 270(14):8367-72); the synthetic peptide based on the amino acid sequence of the C terminal region of native human factor X activation peptide (FXAP) is another example of a linker (Philippou H, et al, An ELISA for factor X activation peptide: application to the investigation of thrombogenesis in cardiopulmonary bypass. British Journal of Haematology, 1995 June, 90(2):432-7); the small antigenic peptide epitope containing part of the hemagglutinin (HA) of influenza virus described by Kast, et al., Biochemistry, 1995 34(13):4402-11); the epsilon-tag peptide of Lehel, et al., PNAS, 1995 92(5):1406-10); the peptide sequence derived and encoded by the tagging insert sequence of Olah, et al., Analytical Biochemistry, 1994 221(1):94-102; the six histidine tag of Sporeno, et al., Production and structural characterization of amino terminally histidine tagged human oncostatin M in E. coli. Cytokine, 1994 6(3):255-64); the streptavidin-affinity tag described by Schmidt, et al., Protein Engineering, 1993 6(1):109-22); and the hemagglutinin epitope sequence described by Pati, Gene, 1992 114(2):285-8). Other linkers are known to those skilled in the art.

The markers identified in Table A have been found to be associated with cells of certain cancers. These markers were identified using the GSX™ system technology as corresponding to genetic suppressor elements that control cell growth. See U.S. provisional patent application Ser. No. 60/381,619, filed May 17, 2002, entitled “Cellular Gene Targets for Controlling Cell Growth” and U.S. patent application Ser. No. 10/441,925, filed May 19, 2003, entitled “Cellular Gene Targets for Controlling Cell Growth,” both incorporated herein by reference in their entirety. Briefly, a Genetic Suppressor Element (GSE), is a gene fragment, which, when expressed in cells, acts as a genetic inhibitor of the corresponding intact gene in those cells. A GSE can exert its effect through either an antisense, or a dominant negative peptide mechanism. GSEs are selected from libraries of DNA fragments, generated by random breakage of sets of test genes, cloned in a retroviral or other expression vector. The RFL clones are introduced into a population of test cells at approximately one test fragment per cell. Cells with a desired new phenotype, resulting from the expression of a GSE, are isolated on the basis of any selectable parameter. The GSEs are recovered from the selected cells and characterized by DNA sequence analysis and further functional assays. See U.S. Pat. No. 5,217,889, issued Jun. 8, 1993, entitled “Methods and applications for efficient genetic suppressor elements” incorporated herein by reference in its entirety.

The markers identified in Table B also have been found to be associated with cells of certain cancers. These markers were identified using the GSX system technology as corresponding to genetic suppressor elements that control cell growth. See U.S. provisional patent application Ser. No. 60/450,886, filed Feb. 26, 2003, entitled “Diagnostic Methods for Cancer Detection” and U.S. patent application Ser. No. 10/789,378, filed Feb. 26, 2004, entitled “Targets for Controlling Cellular Growth and for Diagnostic Methods,” both incorporated herein by reference in their entirety.

The markers identified in Table C also have been found to be associated with cells of certain cancers. See U.S. provisional patent application Ser. No. 60/543,793, filed Feb. 11, 2004, entitled “Compositions And Methods Relating To Angiogenesis And Tumorigenesis.”

The identification of these targets in cancer cells suggests that they may be useful tumor markers for treatment.

A great number of tumor-specific antigens have been identified. For example, a recent review article listed over 50 cancer-associated antigens that are recognized by T-cells. Renkvist N., Castelli C., Robbins P. F., Parmiani G. A listing of human tumor antigens recognized by T cells. Cancer Immunol Immunother. 50, 3-15 (2001). A cell-surface antigen referred to as STEAP (six-transmembrane epithelial antigen of the prostate), for example, has been found to be expressed at high levels in prostate cancer cells but is rarely present in non-prostate tissue. Hubert R S, Vivanco I, Chen E, Rastegar S, Leong K, Mitchell S C, Madraswala R, Zhou Y, Kuo J, Raitano A B, Jakobovits A, Saffran D C, Afar D E. STEAP: a prostate-specific cell-surface antigen highly expressed in human prostate tumors. Proc Natl Acad Sci USA. 96, 14523-8 (1999). Such tumor specific antigens may be used to preferentially target cells of that tumor.

Similarly, certain antigens are known to occur predominantly or exclusively in cells of a particular organ or tissue. Epithelial specific antigen (ESA), for example, is used as a general epithelial cell marker. Such tissue specific antigens may be used to preferentially target cells of that tissue. The invention includes bivalent molecules that are specific for a cancer antigen and an antigen characteristic of a particular organ (i.e., a tumor-specific antigen and a tissue specific antigen).

In some embodiments of the present invention, at least one of the binding moieties is capable of binding a target selected from the group consisting of the markers listed in Table A. In other embodiments, at least one of the binding moieties is capable of binding a target selected from the group consisting of the markers listed in Table B. In other embodiments, at least one of the binding moieties is capable of binding a target selected from the group consisting of the markers listed in Table C. In such embodiments, the other binding moiety can be capable of binding to another cell-surface protein, antigen, or marker that is expressed on cancer cells, a tissue specific protein, antigen, or marker or a tumor specific protein, antigen, or marker. In other embodiments, one of the binding moieties is capable of binding a target selected from the group consisting of the markers listed in Table A, and the other binding moiety is selected from the group consisting of the markers listed in Table A. In other embodiments, one of the binding moieties is capable of binding a target selected from the group consisting of the markers listed in Table A, and the other binding moiety is selected from the group consisting of the markers listed in Table B. In other embodiments, one of the binding moieties is capable of binding a target selected from the group consisting of the markers listed in Table A, and the other binding moiety is selected from the group consisting of the markers listed in Table C. In other embodiments, one of the binding moieties is capable of binding a target selected from the group consisting of the markers listed in Table B, and the other binding moiety is selected from the group consisting of the markers listed in Table B. In other embodiments, one of the binding moieties is capable of binding a target selected from the group consisting of the markers listed in Table B, and the other binding moiety is selected from the group consisting of the markers listed in Table C. In other embodiments, one of the binding moieties is capable of binding a target selected from the group consisting of the markers listed in Table C, and the other binding moiety is selected from the group consisting of the markers listed in Table C.

As discussed above, the binding of the binding moieties and the targeted antigens is preferably of only moderate affinity. Ideally, the avidity of the bivalent molecule for the antigen is sufficiently high only when both targeted antigens are present on the surface of a cancer cell. This further decreases the likelihood that the bivalent molecule will become associated with a cell expressing only one of the antigens. For purposes of this application, “moderate affinity” means an affinity in the range of about 10 nM to 100 μM.

The binding moieties of moderate affinity may be identified by screening libraries and by phage display. In addition, moderate affinity variants can be prepared from binding moieties of higher affinity by a number of methods known in the art including the random or site-directed mutagenesis of the high-affinity binding moiety.

The bivalent molecules of the present invention are useful for treating cancer in patients in need of such treatment. Any of the bivalent molecules of the invention can be used, subject to such considerations as variations in bioavailability, antigenicity, and potency among the different bivalent molecules. The amount and method of administering the therapeutic molecules of the invention can be ascertained by one skilled in the art. In some embodiments, administration of large bivalent molecules is by injection, whether subcutaneous, intramuscular, or intravenous. Smaller, less peptidic bivalent molecules can be administered orally as well as intravenously. For example, nonprotein containing bivalent molecules may be useful where stability or the need for an orally active drug is an issue.

Cancers that can be targeted and treated in accordance with the present invention include carcinomas, melanomas, sarcomas, neuroblastomas, leukemias, lymphomas, gliomas myelomas, breast cancers, colon cancers, lung cancers, renal cancers, ovarian cancers, prostate cancers, and uterine cancers. The bivalent molecules may be used to treat primary tumors or metastatic disease. As described above, the optimum radioisotope to be used as the therapeutic agent may depend upon the size of the tumor as well as the penetration of the bivalent molecule. For optimum tumor eradication, the combination of radioisotope and physical penetration of the bivalent molecule should be matched so that the radioisotope is able to deliver effective toxicity to cells throughout the tumor.

The invention also provides a bivalent molecule that comprises an imaging agent and two binding moieties, each binding moiety being specific for a different antigen produced by or associated with cancer cells (e.g., a cell-surface antigen). The invention also provides a method for diagnosing, staging and imaging cancer by administering the bivalent molecule to a patient and subsequently measuring the location, extent or distribution of the imaging agent.

The bivalent molecule used for diagnosis, staging and imaging is as described above except that isotopes or other imaging agents are selected for their ability to be detected rather than for their therapeutic value. Imaging agents include imaging contrast agents, such as gadolinium, ultrasound imaging agents, and nuclear imaging agents, such as Tc-99m, In-111, Ga-67, Rh-105, 1-123, Nd-147, Pm-151, Sm-153, Gd-159, Th-161, Er-171, Re-186, Re-188, and Tl-201. For in vivo use, the scope of the invention includes any isotope known in the art for imaging; of these, Tc-99m and In-111 are exemplary.

The bivalent imaging agents of the present invention are useful in imaging a patient generally, and/or in specifically detecting or diagnosing the presence of diseased tissue in a patient. The imaging process may be carried out by administering an imaging agent of the invention to a patient, and then scanning the patient using ultrasound or magnetic resonance imaging to obtain visible images of an internal region of a patient and/or of any diseased tissue in that region. By region of a patient, it is meant the whole patient, or a particular area or portion of the patient. The imaging contrast agent may be employed to provide images of the vasculature, heart, liver, and spleen, and in imaging the gastrointestinal region or other body cavities, or in other ways as will be readily apparent to those skilled in the art, such as in tissue characterization, blood pool imaging, etc. Any of the various types of ultrasound or magnetic resonance imaging devices can be employed in the practice of the invention, the particular type or model of the device not being critical to the method of the invention.

Detection may also be performed in vitro on a tissue section or other sample. In such embodiments, other indicators that are well-known in the art may be used (e.g., horseradish peroxidase).

In a typical protocol, a series of single-photon emission tomography (SPET) images are taken of a patient after administration of the radiolabeled bivalent molecule. Comparison of the images will reveal the location of the radiolabel. Initial views may show the label distributed in blood vessels and vascular tissue, but later views will show concentration of the label at the targeted tumor(s). Finding the presence of concentrated label will allow a diagnosis to be made, either based on the images alone or in combination with other factors (e.g., results of a subsequent biopsy). Determining the location and extent of the tumor(s) will allow staging of the cancer.

A “therapeutically effective amount” means an amount effective to either (1) reduce the symptoms of the disease sought to be treated or (2) induce a pharmacological change relevant to treating the disease sought to be treated. For cancer, an effective amount includes an amount effective to: reduce the size of a tumor; slow the growth of a tumor; prevent or inhibit metastases; or increase the life expectancy of the affected animal.

Therapeutically effective amounts of the therapeutic agents can be any amount or doses sufficient to bring about the desired effect and depend, in part, on the condition, type and location of the cancer, the size and condition of the patient, as well as other factors readily known to those skilled in the art. The dosages can be given as a single dose, or as several doses, for example, divided over the course of several weeks.

The present invention is also directed toward methods of treatment utilizing the therapeutic compositions of the present invention. The method comprises administering the therapeutic agent to a subject in need of such administration.

The invention also provides a pharmaceutical composition comprising a therapeutically effective amount of the bivalent molecule of the invention in a pharmaceutically acceptable adjuvant, which can be selected from one or more of a pharmaceutically acceptable excipient, diluent, carrier, preservative, emulsifier, anti-oxidant and/or stabilizer. Such components are known to one skilled in the art. See, e.g., Remington's Pharmaceutical Sciences, 18th ed. A. R. Gennaro, ed. Mack, Easton, Pa. (1990). Thus, for example, the pharmaceutical composition of the present invention may be provided in vials containing an appropriate concentration of the drug in sterile 0.9% sodium chloride solution for injection. Examples of other excipients include water, saline, Ringer's solution, dextrose solution, mannitol, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, Tris buffer, histidine, citrate, and glycine, or mixtures thereof, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

One embodiment of the present invention is a controlled release formulation that is capable of slowly releasing a composition of the present invention into an animal. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

The bivalent molecules of the instant invention can be administered by any suitable means, including, for example, parenteral, topical, oral or local administration, such as intradermally, by injection, or by aerosol. In one embodiment of the invention, the bivalent molecule is administered by injection. Such injection can be locally administered to any affected area. A therapeutic composition can be administered in a variety of unit dosage forms depending upon the method of administration. For example, unit dosage forms suitable for oral administration of an animal include powder, tablets, pills and capsules. Preferred delivery methods for a therapeutic composition of the present invention include intravenous administration and local administration by, for example, injection or topical administration. For particular modes of delivery, a therapeutic composition of the present invention can be formulated in an excipient of the present invention. A therapeutic composition of the present invention can be administered to any animal, preferably to mammals, and more preferably to humans.

The particular mode of administration will depend on the condition to be treated. It is contemplated that administration of the agents of the present invention may be via any bodily fluid, or any target or any tissue accessible through a body fluid.

Preferred routes of administration of cell-surface targeted bivalent molecules of the present invention are by intravenous, interperitoneal, or subcutaneous injection including administration to veins or the lymphatic system. As indicated above, a targeted agent can be designed to focus on markers present in other fluids, body tissues, and body cavities, e.g. synovial fluid, ocular fluid, or spinal fluid. Thus, for example, an agent can be administered to spinal fluid, where an antibody targets a site of pathology accessible from the spinal fluid. Intrathecal delivery, that is, administration into the cerebrospinal fluid bathing the spinal cord and brain, may be appropriate for example, in the case of a target residing in the choroid plexus endothelium of the cerebral spinal fluid (CSF)-blood barrier.

All references cited herein are fully incorporated by reference.

TABLE A

| | REFERENCE |
|---|---|
| TARGET | NUMBER |
| amyloid beta (A4) precursor-like protein 2 (amyloid precursor | XM_050724 |
| protein homolog HSD-2, APPH; APPL2; CDEBP; APLP2) | |
| amyloid beta (A4) precursor protein (amyloid beta-peptide; APP, | NM_000484 |
| AAA; ABETA; AD1; CVAP) | |
| Adenocarcinoma antigen recognized by T lymphocytes-4 (ART-4) | NM_014062 |
| bone marrow stromal cell antigen 2 (BST2) | NM_004335 |
| CD63 (granulophysin; lysosome-associated membrane glycoprotein | NM_001780 |
| 3; melanoma 1 antigen; melanoma-associated antigen ME491; | |
| melanoma-associated antigen MLA1; ocular melanoma-associated | |
| antigen, MP-3; ME491; MLA1; OMA81H) | |
| CD81 (26 kDa cell surface protein TAPA-1; target of | NM_004356 |
| antiproliferative antibody 1; 7; TAPA-1; TAPA1) | |
| carcinoembryonic antigen-related cell adhesion molecule 5 | NM_004363 |
| (carcinoembryonic antigen; CD66e; CEA; CEACAM5) | |
| v-kit Hardy-Zuckerman 4 feline sarcoma viral oncogene homolog | NM_000222 |
| (CD117; PBT; SCFR KIT, C-KIT) | |
| collagen, type iv, alpha-2 (COL4A2) | XM_049904 |
| diazepam binding inhibitor (GABA receptor modulator, acyl- | M15887 |
| Coenzyme A binding protein. Endozepine; ACBP; DBI) | |
| Ephrin A1 (eph tyrosine kinase 1; eph tyrosine kinase 1 | NM_004428 |
| (erythropoietin-producing hepatoma amplified sequence; eph | |
| tyrosine kinase 1 (erythropoietin-producing hepatoma amplified | |
| sequence); ephrin receptor EphA1; ephrin receptor EphA1); ephrin | |
| type-A receptor 1; erythropoietin-producing hepatoma amplified | |
| sequence; oncogene EPH; tyrosine-protein kinase receptor EPH; | |
| EPH; EPHT; EPHT1; EFNA1) | |
| ENSA (alpha endosulfine, endosulfine alpha) | XM_041911 |
| fibrillin 2 (CCA, FBN2) | NM_001999 |
| fibroblast growth factor receptor 1 (fms-related tyrosine kinase 2; | NM_000604 |
| FLG protein; FMS-like tyrosine kinase 2; N-sam tyrosine kinase; | |
| basic fibroblast growth factor receptor 1; fms-related tyrosine kinase- | |
| 2; heparin-binding growth factor receptor; hydroxyaryl-protein | |
| kinase; protein-tyrosine kinase; tyrosylprotein kinase; BFGFR; C- | |
| FGR; CEK; FLG; FLJ14326; FLT2; H2; H3; H4; H5; KAL2; N- | |
| SAM FGFR1) | |
| fibulin 6 (FIBL-6) | XM_053531 |
| growth factor, augmenter of liver regeneration (ERV1 homolog, S. cerevisiae; | NM_005262 |
| Growth factor, erv1-like (augmenter of liver regeneration; | |
| growth factor, erv1 (S. cerevisiae)-like (augmenter of liver | |
| regeneration); hepatic regenerative stimulation substance; | |
| hepatopoietin; ALR; ERV1; HERV1; HPO1; HPO2; HSS; GFER) | |
| hepatoma-derived growth factor (HMG1L2; HDGF) | NM_004494 |
| intercellular adhesion molecule 2 (CD102, ICAM2) | NM_000873 |
| insulin-like growth factor 2 receptor (mannose-6-phosphate receptor, | NM_000876 |
| cation-independent; cation-independent mannose-6 phosphate | |
| receptor; CD222; CIMPR; M6P-R; MPRI, IGF2R) | |
| IK cytokine (IK factor; RD element; chondrosarcoma-associated | NM_006083 |
| protein 2; prer protein CSA2; RED; IK) | |
| interleukin 4 receptor (CD124; IL4RA; IL4R) | NM_000418 |
| alpha 3 integrin (antigen CD49C, alpha 3 subunit of VLA-3 receptor; | NM_005501 |
| CD49C; CD49c; GAPB3; VL3A ITGA3) | |
| alpha 4 integrin (antigen CD49D, alpha 4 subunit of VLA-4 receptor; | NM_000885 |
| CD49D; CD49d; ITGA4) | |
| beta 5 integrin, ITGB5 | X53002 |
| L1 cell adhesion molecule (antigen identified by monoclonal | NM_000425 |
| antibody R1; neural cell adhesion molecule L1; CAML1; CD171; | |
| HSAS; HSAS1; MASA; MIC5; N-CAML1; S10; SPG1, L1CAM) | |
| neuropilin 1 (NRP; VEGF165R, NRP1) | AF018956 |
| PDGFA associated protein 1 (PDGF associated protein; HASPP28; | NM_014891 |
| PAP; PAP1; PDAP1) | |
| prion protein (p27-30) (CJD; GSS; PRIP; PrP; PrPc, PRNP) | NM_000311 |
| alpha prothymosin (TMSA; PTMA) | NM_002823 |
| silver homolog (mouse) (melanocyte protein mel 17; Pmel 17; | BC001414 |
| Pmel17; D12S53E; ME20; PMEL17; Pmel 17; Pmel17; SI; SIL; | |
| gp 100; SILV) | |
| solute carrier family 31 (copper transporters), member 1 (human | NM_001859 |
| high-affinity copper uptake protein (hCTR1); copper transporter 1; | |
| hCTR1; COPT1; CTR1; U83460.1; SLC31A1) | |
| solute carrier family 5 (sodium-dependent vitamin transporter), | NM_021095 |
| member 6 (SMVT; SLC5A6) | |
| stanniocalcin 2 (stanniocalcin related protein; stanniocalcin 2; STC- | XM_037313 |
| 2; STCRP; STC2) | |
| teratocarcinoma-derived growth factor 1 (CR; CRGF; CRIPTO; | NM_003212 |
| TDGF1) | |
| von Willebrand factor (Coagulation factor VIII; F8VWF; VWF) | NM_000552 |
| xenotropic and polytropic retrovirus receptor (SYG1; X3; XPR1) | NM_004736 |
| calmodulin binding protein 4 (striatin; STRN4; ZIN) | NM_013403 |
| PCTAIRE protein kinase 1, transcript variant 1, PCTK1 | NM_006201 |

TABLE B

| Accession | Common | |
|---|---|---|
| Number | Name | Description |
| NM_001087 | AAMP | angio-associated, migratory cell protein |
| NM_001109 | ADAM8 | a disintegrin and metalloproteinase domain 8 |
| NM_139057 | ADAMTS17 | a disintegrin-like and metalloprotease (reprolysin type) with |
| | | thrombospondin type 1 motif, 17 |
| NM_004036 | ADCY3 | adenylate cyclase 3 |
| NM_001619 | ADRBK1 | adrenergic, beta, receptor kinase 1 |
| NM_006698 | BLCAP | bladder cancer associated protein |
| NM_012264 | C22orf5 | chromosome 22 open reading frame 5 |
| NM_004356 | CD81 | CD81 antigen (target of antiproliferative antibody 1) |
| NM_001769 | CD9 | CD9 antigen (p24) |
| NM_001305 | CLDN4 | claudin 4 |
| NM_001288 | CLIC1 | chloride intracellular channel 1 |
| NM_058175 | COL6A2 | collagen, type VI, alpha 2 |
| AF070636 or | CTL2 | CTL2 gene |
| NM_020428 | | |
| NM_001397 | ECE1 | endothelin converting enzyme 1 |
| NM_004429 | EFNB1 | ephrin-B1 |
| NM_004475 | FLOT2 | flotillin 2 |
| AC011511 or | ICAM3 | intercellular adhesion molecule 3 |
| BC058903 | | |
| NM_006123 | IDS | iduronate 2-sulfatase (Hunter syndrome) |
| NM_002226 | JAG2 | jagged 2 |
| BC001699 | JAM1 | junctional adhesion molecule 1 |
| NM_005567 | LGALS3BP | lectin, galactoside-binding, soluble, 3 binding protein |
| XM_085426 | LOC146330 | similar to possible G-protein receptor |
| BC020590 | LOC51107 | CGI-78 protein |
| NM_000237 | LPL | lipoprotein lipase |
| NM_002335 | LRP5 | low density lipoprotein receptor-related protein 5 |
| NM_005581 | LU | Lutheran blood group (Auberger b antigen included) |
| NM_005898 | M11S1 | membrane component, chromosome 11, surface marker 1 |
| NM_007061 | MSE55 | serum constituent protein |
| NM_006702 | NTE | neuropathy target esterase |
| AK055605 or | PLXNA1 | Homo sapiens cDNA FLJ31043 fis, clone HSYRA2000248 |
| AK126101 | | (PLEXIN A1) or Homo sapiens cDNA FLJ44113 fis, clone |
| | | TESTI4046487, highly similar to Mus musculus plexin A1 |
| AF034800 | PPFIA3 | protein tyrosine phosphatase, receptor type, f polypeptide |
| | | (PTPRF), interacting protein (liprin), alpha 3 |
| NM_145648 | PTR4 | Homo sapiens peptide-histidine transporter 4 (PTR4), |
| | | mRNA |
| NM_004207 | SLC16A3 | solute carrier family 16 (monocarboxylic acid transporters), |
| | | member 3 |
| NM_005628 | SLC1A5 | solute carrier family 1 (neutral amino acid transporter), |
| | | member 5 |
| NM_014437 | SLC39A1 | solute carrier family 39 (zinc transporter), member 3 |
| NM_021102 | SPINT2 | serine protease inhibitor, Kunitz type, 2 |
| NM_003714 | STC2 | stanniocalcin 2 |
| NM_014452 | TNFRSF21 | tumor necrosis factor receptor superfamily, member 21 |
| NM_003299 | TRA1 | tumor rejection antigen (gp96) 1 |
| NM_017636 | TRPM4 | transient receptor potential cation channel, subfamily M, |
| | | member 4 |

TABLE C

| Common Name | Description |
|---|---|
| LAMB3 | laminin, beta 3 (nicein (125 kD); kalinin (140 kD), BM600 |
| | (125 kD)); BM600-125 kDa; LAMNB1; kalinin-140 kDa; nicein-125 kDa |
| ANGPTL4 | angiopoietin-like 4; Alternate Names: PPARG |
| | angiopoietin related protein; fasting-induced adipose |
| | factor; hepatic angiopoietin-related protein; hepatic |
| | fibrinogen/angiopoietin-related protein ; ANGPTL2; |
| | ARP4; FIAF; HFARP; PGAR; PP1158; PPARG; pp1158 |
| COL1A1 | collagen, type I, alpha 1; Alternate Names: Collagen I, |
| | alpha-1 polypeptide; collagen of skin, tendon and bone, |
| | alpha-1 chain; osteogenesis imperfecta type IV; OI4 |
| PCDH89 | |
| GPR57 | G protein-coupled receptor 57 |
| GP2 | glycoprotein 2 (zymogen granule membrane); pancreatic |
| | zymogen granule membrane associated protein GP2 beta form; ZAP75 |
| PTGER3 | prostaglandin E receptor 3 (subtype EP3); Prostaglandin E |
| | receptor 3, EP3 subtype; EP3 |
| KITLG | KIT ligand; mast cell growth factor; stem cell factor |
| | precursor; KITL; KL-1; Kitl; MGF; SCF; SF |
| RAP2B | RAP2B, member of RAS oncogene family |
| COL5A1 | collagen, type V, alpha 1 |
| SEPP1 | selenoprotein P, plasma, 1; SeP |
| CXCL1 | chemokine (C-X-C motif) ligand 1 (melanoma growth |
| | stimulating activity, alpha); GRO1 oncogene (melanoma |
| | growth stimulating activity, alpha); GRO1 oncogene |
| | (melanoma growth-stimulating activity); GRO1; GROA; |
| | GROa; MGSA; MGSA-a; NAP-3; SCYB1 |
| TNC | tenascin C (hexabrachion); Hexabrachion (tenascin); |
| | hexabrachion (tenascin C, cytotactin); HXB; TN |
| LTBP1 | latent transforming growth factor beta binding protein 1 |
| PRSS11 | protease, serine, 11 (IGF binding); HTRA; HTRA1; HtrA; L56; ORF480 |
| FN1 | fibronectin 1; cold-insoluble globulin; CIG; FINC; FN; |
| | LETS |
| FZD1 | fizzled homolog 1 (Drosophila); Frizzled, drosophila, |
| | homolog of, 1; Wnt receptor |
| SPP1 | secreted phosphoprotein 1 (osteopontin, bone sialoprotein |
| | I, early T-lymphocyte activation 1); Secreted |
| | phosphoprotein-1 (osteopontin, bone sialoprotein); BNSP; |
| | BSPI; ETA-1; OPN |
| IGFBP7 | insulin-like growth factor binding protein 7; FSTL2; |
| | IGFBP-7; MAC25; PSF |
| RNASE4 | ribonuclease, RNase A family, 4; RNS4 |
| SCHODL | |
| NOV | nephroblastoma overexpressed gene; CCN3; IGFBP9; NOVH |
| COL12A1 | collagen, type XII, alpha 1; BA209D8.1; DJ234P15.1 |
| MAGP2 | microfibril-associated glycoprotein 2 |
| GPR23 | G protein-coupled receptor 23; P2Y5-LIKE; P2Y9 |
| TLL1 | tolloid-like 1; TLL |
| GPR44 | G protein-coupled receptor 44; chemoattractant receptor- |
| | homologous molecule expressed on TH2 cells; CRTH2 |
| MGC2376 | potassium channel tetramerisation domain containing 14 (KCTD14) |
| NPY1R | neuropeptide Y receptor Y1; Neuropeptide Y receptor; |
| | NPYR |
| EMP3 | epithelial membrane protein 3; YMP |
| HLA-A | major histocompatibility complex, class II, DO alpha; |
| | HLA-D0-alpha; lymphocyte antigen; major |
| | histocompatibility complex, class II, DN alpha; HLA-D0- |
| | alpha; HLA-DNA; HLA-DZA; HLADZ |
| GNAO1 | guanine nucleotide binding protein (G protein), alpha |
| | activating activity polypeptide O; G-ALPHA-o; GNAO |
| CCR5 | chemokine (C-C motif) receptor 5; chemokine (C-C) |
| | receptor 5; chemr13; CC-CKR-5; CCCKR5; CKR-5; CKR5; |
| | CMKBR5 |
| C20orf52 | chromosome 20 open reading frame 52; homolog of mouse |
| | RIKEN 2010100O12 gene; bA353C18.2 |
| SORCS3 | VPS10 domain receptor protein; KIAA1059, SORCS |
| PF4 | platelet factor 4; platelet factor 4; CXCL4; SCYB4 |
| SPINK2 | serine protease inhibitor, Kazal type, 2 (acrosin-trypsin |
| | inhibitor); HUSI-II |
| 1GSF6 | |
| GPR110 | G protein-coupled receptor 110; G-protein coupled |
| | receptor 110; hGPCR36 |
| OR1J5 | olfactory receptor, family 1, subfamily J, member 5; HSA5 |
| BGLAP | bone gamma-carboxyglutamate (gla) protein (osteocalcin); |
| | Bone gamma-carboxyglutamic acid protein; osteocalcin; |
| | BGP |
| GALR2 | galanin receptor 2; GALNR2 |
| HCN2 | hyperpolarization activated cyclic nucleotide-gated |
| | potassium channel 2; brain cyclic nucleotide gated channel |
| | 2; BCNG-2; BCNG2; HAC-1 |
| CD81 | CD81 antigen (target of antiproliferative antibody 1); 26 kDa |
| | cell surface protein TAPA-1; target of antiproliferative antibody 1; |
| | S5.7; TAPA-1; TAPA1 |
| OGFR | opioid growth factor receptor; 7-60 protein; zeta-type |
| | opioid receptor; 7-60; Jul-60 |
| GPR6 | G protein-coupled receptor 6 |
| OMP | olfactory marker protein; Olfactory marker protein |
| CMA1 | chymase 1, mast cell; chymase, heart; chymase, mast cell; |
| | mast cell protease I; CYH; MCT1 |
| DKFZP564D0 | |
| CHRM1 | cholinergic receptor, muscarinic 1; muscarinic |
| | acetylcholine receptor M1; HM1; M1 |
| PYY | peptide YY |
| FGF19 | fibroblast growth factor 19 |
| AGTR2 | angiotensin II receptor, type 2; angiotensin receptor 2; |
| | AT2 |
| SSTR3 | somatostatin receptor 3 |
| TMPO | thymopoietin; LAP2; TP |
| TAS2R16 | taste receptor, type 2, member 16; candidate taste receptor T2R16; T2R16 |
| ADORA2B | adenosine A2b receptor; ADORA2 |
| GPR10 | G protein-coupled receptor 10; prolactin releasing peptide |
| | receptor; prolactin-releasing hormone receptor; GR3; |
| | PrRPR |
| ADCYAP1R1 | adenylate cyclase activating polypeptide 1 (pituitary) |
| | receptor type I; adenylate cyclase activating polypeptide 1 |
| | (pituitary) receptor type 1; PACAPR; PACAPRI |
| OR1F10 | olfactory receptor, family 1, subfamily F, member 10; OR3-145 |
| HDGF | hepatoma-derived growth factor (high-mobility group |
| | protein 1-like); Hepatoma-derived growth factor; |
| | HMG1L2 |
| CD151 | CD151 antigen; hemidesmosomal tetraspanin CD151; |
| | membrane glycoprotein SFA-1; platelet surface |
| | glycoprotein gp27; platelet-endothelial cell tetraspan |
| | antigen 3; GP27; PETA-3; SFA-1; SFA1 |
| PDAP1 | PDGFA associated protein 1; PDGF associated protein; |
| | HASPP28; PAP; PAP1 |
| A1BG | alpha-1-B glycoprotein; A1B; ABG; GAB |
| LIPF | lipase, gastric; HGL; HLAL |
| PBEF | pre-B-cell colony-enhancing factor |
| C1QTNF3 | C1q and tumor necrosis factor related protein 3; |
| | collagenous repeat-containing sequence of 26-kDa; |
| | complement-c1q tumor necrosis factor-related protein 3; |
| | CORS26; CTRP3; FLJ37576 |
| SLC39A4 | solute carrier family 39 (zinc transporter), member 4; FLJ20327; |
| | ZIP4 |
| IFNGR2 | interferon gamma receptor 2 (interferon gamma transducer |
| | 1); interferon gamma receptor accessory factor-1; |
| | interferon-gamma receptor beta chain precursor; AF-1; |
| | IFGR2; IFNGT1 |
| ENT3 | solute carrier family 29 (nucleoside transporters), member |
| | 3 (SLC29A3); FLJ11160 |
| SERPINC1 | serine (or cysteine) proteinase inhibitor, clade C |
| | (antithrombin), member 1; antithrombin III; AT3; ATIII; |
| | antithrombin III |
| NRP1 | neuropilin 1; NRP; VEGF165R |
| CACNA1H | calcium channel, voltage-dependent, alpha 1H subunit; |
| | calcium channel, voltage-dependent, T type, alpha 1Hb subunit; |
| | CACNA1HB |
| CD44 | CD44 antigen (homing function and Indian blood group |
| | system); CD44 antigen (homing function); CD44R; IN; |
| | MC56; MDU2; MDU3; MIC4; Pgp1 |
| STC2 | stanniocalcin 2; stanniocalcin related protein; stanniocalcin |
| | 2; stanniocalcin related protein; STC-2; STCRP |
| DLK1 | delta-like 1 homolog (Drosophila); FA1; PG2; PREF-1; |
| | PREF1; Pref-1; ZOG; pG2 |
| F2R | coagulation factor II (thrombin) receptor; protease- |
| | activated receptor 1; thrombin receptor; CF2R; PAR1; TR |
| EMP2 | epithelial membrane protein 2; XMP |
| HBE1 | hemoglobin, epsilon 1 |
| BSG | basigin (OK blood group); M6 antigen; OK blood group; |
| | collagenase stimulatory factor; emmprin; extracellular |
| | matrix metalloproteinase inducer; 5F7; CD147; |
| | EMMPRIN; HGNC: 8130; M6; OK; TCSF |
| GPR80 | G protein-coupled receptor 80; G protein-coupled receptor |
| | 99; P2Y-like nucleotide receptor; GPR99; HGNC: 14591 |
| APOB48R | macrophage receptor for apolipoprotein B48 |
| AMELY | amelogenin (Y chromosome); AMGL; AMGY |
| IL26 | interleukin 26; AK155 protein (AK155 gene); AK155; IL- |
| | 26 |
| TRPM5 | transient receptor potential cation channel, subfamily M, |
| | member 5; MLSN1 and TRP-related; MLSN1-and TRP-related; LTRPC5; |
| | MTR1 |
| ENSA | endosulfine alpha; alpha endosulfine |
| OR1F1 | olfactory receptor, family 1, subfamily F, member 1; |
| | Olfmf; olfactory receptor, family 1, subfamily F, member |
| | 4; olfactory receptor, family 1, subfamily F, member 5; |
| | olfactory receptor, family 1, subfamily F, member 6; |
| | olfactory receptor, family 1, subfamily F, member 7; |
| | olfactory receptor, family 1, subfamily F, member 8; |
| | olfactory receptor, family 1, subfamily F, member 9; |
| | HGNC: 8198; HGNC: 8199; HGNC: 8200; HGNC: 8201; |
| | HGNC: 8202; HGNC: 8203; OLFMF; OR16-36; OR16-37; |
| | OR16-88; OR16-89; OR16-90; OR1F4; OR1F5; OR1F6; |
| | OR1F7; OR1F8; OR1F9; Olfmf |
| GP3ST | betaGal-3-O-sulfotransferase |
| BDNF | brain-derived neurotrophic factor; MGC34632 |
| PLXN3 | plexin A3; 6.3; Sex chromosome X transmembrane protein |
| | of HGF receptor family 3; plexin 4; 6.3; PLEXIN-A3; |
| | PLXN3; PLXN4; Plxn3; SEX; XAP-6 |
| APMCF1 | APMCF1 protein (non-HGNC gene) |
| SCAMP1 | secretory carrier membrane protein 1; SCAMP; SCAMP37 |
| PALMD | palmdelphin; chromosome 1 open reading frame 11; |
| | paralemnin-like; C1orf11; FLJ20271; HGNC: 1231; |
| | PALML |
| MMP8 | matrix metalloproteinase 8 (neutrophil collagenase); |
| | PMNL collagenase; neutrophil collagenase; CLG1; HNC; PMNL-CL |
| MFAP3 | microfibrillar-associated protein 3 |
| SPAG11 | sperm associated antigen 11; epididymal protein 2; sperm |
| | associated antigen 11 precursor; EP2; EP2C; EP2D; HE2 |
| A2M | alpha-2-macroglobulin |
| NET-2 | transmembrane 4 superfamily member 12; tetraspan NET-2 |
| CXCL11 | chemokine (C-X-C motif) ligand 11; small inducible |
| | cytokine subfamily B (Cys-X-Cys), member 11; small |
| | inducible cytokine subfamily B (Cys-X-Cys), member 9B; |
| | B-R1; H174; I-TAC; IP-9; IP9; SCYB11; SCYB9B; b-R1 |
| KLRB1 | killer cell lectin-like receptor subfamily B, member 1; |
| | hNKR-P1A; CD161; NKR; NKR-P1; NKR-P1A; |
| | NKRP1A; hNKR-P1A |
| TF | transferrin; PRO1557 |
| COL14A1 | collagen, type XIV, alpha 1 (undulin); collagen, type XIV, |
| | alpha 1; undulin; undulin (fibronectin-tenascin-related); |
| | UND |
| IL7 | interleukin 7; IL-7 |
| COL9A1 | collagen, type IX, alpha 1; cartilage-specific short |
| | collagen; collagen IX, alpha-1 polypeptide; DJ149L1.1.2; |
| | MED |
| CCR4 | chemokine (C-C motif) receptor 4; chemokine (C-C) |
| | receptor 4; CC-CKR-4; CKR4; CMKBR4; ChemR13; HGCN: 14099; |
| | K5-5; k5-5 |
| FPR1 | formyl peptide receptor 1; FMLP; FPR |
| FAP | fibroblast activation protein, alpha; integral membrane |
| | serine protease; seprase; DPPIV; FAPA; SEPRASE |
| OPCML | opioid binding protein/cell adhesion molecule-like; opiate |
| | binding-cell adhesion molecule; opioid-binding |
| | protein/cell adhesion molecule-like; OBCAM; OPCM |
| GPR145 | Melanin-concentrating hormone receptor 2; MCH receptor |
| | 2; MCHR-2; MCH-R2; MCH2R; MCH-2R; MCH2; G |
| | protein coupled receptor 145 |
| GFRA3 | GDNF family receptor alpha 3; GFRalpha3; GPI-linked |
| | receptor; glial cell line-derived neurotrophic factor |
| | receptor alpha-3; GFRA-3; GFRa-3 |
| EDN3 | endothelin 3; ET3 |
| IL12B | interleukin 12B (natural killer cell stimulatory factor 2, |
| | cytotoxic lymphocyte maturation factor 2, p40); IL12, |
| | subunit p40; IL23, subuint p40; cytotoxic lymphocyte |
| | maturation factor 2, p40; interkeukin-12 beta chain; |
| | interleukin 12, p40; interleukin 12B; interleukin-12 beta |
| | chain; natural killer cell stimulatory factor, 40 kD subunit; |
| | natural killer cell stimulatory factor-2; CLMF; CLMF2; |
| | IL-12B; NKSF; NKSF2 |
| CXCR4 | chemokine (C-X-C motif), receptor 4 (fusin); |
| | Neuropeptide Y receptor Y3; chemokine (C-X-C motif), |
| | receptor 4 (fusin); D2S201E; HM89; HSY3RR; LAP3; LESTR; NPY3R; |
| | NPYR; NPYY3R; WHIM; fusin |
| PCSK5 | proprotein convertase subtilisin/kexin type 5; prohormone |
| | convertase 5; proprotein convertase PC5; protease PC6; |
| | subtilisin/kexin-like protease PC5; PC5; PC6; PC6A; |
| | SPC6 |
| NID2 | nidogen 2; nidogen 2; nidogen 2 (osteonidogen) |
| ITGA4 | integrin, alpha 4 (antigen CD49D, alpha 4 subunit of VLA- |
| | 4 receptor); antigen CD49D, alpha-4 subunit of VLA-4 |
| | receptor; CD49D; CD49d |
| KIAA1870 | unidentified protein from brain |
| FBLN5 | fibulin 5; developmental arteries and neural crest |
| | epidermal growth factor-like; urine p50 protein; DANCE; EVEC; UP50 |
| TRPV2 | transient receptor potential cation channel, subfamily V, |
| | member 2; vanilloid receptor-like protein 1; MGC12549; |
| | VRL; VRL-1; VRL1 |
| FGF23 | fibroblast growth factor 23; Hypophosphatemia vitamin D- |
| | resistant rickets-2 (autosomal dominant); tumor-derived |
| | hypophophatemia inducing factor; ADHR; HPDR2; HYPF |
| TEM5 | tumor endothelial marker 5 precursor |
| CR1 | complement component (3b/4b) receptor 1, including |
| | Knops blood group system; C3-binding protein; CD35 |
| | antigen; complement component (3b/4b) receptor-1; C3BR; CD35 |
| GPA33 | glycoprotein A33 (transmembrane); A33 |
| CLCA4 | chloride channel, calcium activated, family member 4; |
| | CACC2; CaCC; CaCC2 |
| TIMP3 | tissue inhibitor of metalloproteinase 3 (Sorsby fundus |
| | dystrophy, pseudoinflammatory); K222 expressed in |
| | degenerative retinas; Tissue inhibitor of metalloproteinase- |
| | 3; HSMRK222; K222TA2; SFD |
| MMP10 | matrix metalloproteinase 10 (stromelysin 2); stromelysin |
| | 2; transin 2; SL-2; STMY2 |
| FUT8 | fucosyltransferase 8 (alpha (1,6) fucosyltransferase); GDP- |
| | L-Fuc: N-acetyl-beta-D-glucosaminide alpha1,6- |
| | fucosyltransferase; GDP-fucose—glycoprotein |
| | fucosyltransferase; alpha1-6FucT; glycoprotein 6-alpha-L- |
| | fucosyltransferase; MGC26465 |
| V1RL1 | putative pheromone receptor V1RL1 long form |
| EBI2 | Epstein-Barr virus induced gene 2 (lymphocyte-specific G |
| | protein-coupled receptor); Epstein-Barr virus induced gene 2 |
| ADAM28 | a disintegrin and metalloproteinase domain 28; ADAM23; |
| | EMDCII; MDC-LM; MDC-LS; MDC-Lm; MDC-Ls; MDCL; eMDCII |
| GPLD1 | glycosylphosphatidylinositol specific phospholipase D1; |
| | GPI-specific phospholipase D; glycoprotein phospholipase |
| | D; glycosylphosphatIdylinositol-specific phospholipase D; |
| | phospholipase D, phosphatidylinositol-glycan-specific; |
| | GPIPLD; GPIPLDM; MGC22590; PIGPLD; PIGPLD1 |
| CP | ceruloplasmin (ferroxidase); Ceruloplasmin |
| EPHA3 | EphA3; Ephrin receptor EphA3 (human embryo kinase 1); |
| | eph-like tyrosine kinase 1; eph-like tyrosine kinase 1 |
| | (human embryo kinase 1); ephrin receptor EphA3; human |
| | embryo kinase 1; ETK; ETK1; HEK; HEK4; TYRO4 |
| KLK11 | kallikrein 11; hippostasin; protease, serine, 20 trypsin-like; |
| | protease, serine, trypsin-like; MGC33060; PRSS20; TLSP |
| OR7A17 | olfactory receptor, family 7, subfamily A, member 17; HTPCRX19 |
| IFI27 | interferon, alpha-inducible protein 27; P27 |
| RNASE6 | ribonuclease, RNase A family, k6; RNASEK6; RNS6; |
| | RNase; RNase k6; RNasek6; k6 |
| SELPLG | selectin P ligand; CD162; PSGL-1; PSGL1 |
| CST7 | cystatin F (leukocystatin); cystatin 7; cystatin-like |
| | metastasis-associated protein; leukocystatin; CMAP |
| LEC3 | latrophilin 3 (LPHN3); KIAA0768 |
| TSHR | thyroid stimulating hormone receptor; thyrotropin receptor |
| MC2R | melanocortin 2 receptor (adrenocorticotropic hormone); |
| | Melanocortin-2 receptor (ACTH receptor); melanocortin 2 |
| | receptor (adrenocorticotropic hormone receptor); ACTHR |
| SV2 | synaptic vesicle glycoprotein 2A; synaptic vesicle |
| | glycoprotein 2; KIAA0736; SV2 |
| SERPINA4 | serine (or cysteine) proteinase inhibitor, clade A (alpha-1 |
| | antiproteinase, antitrypsin), member 4; protease inhibitor 4 (kallistatin); KAL; |
| | KLST; KST; PI4; kallistatin |
| ANGPT2 | angiopoietin 2; Ang2; ANG2; Ang2 |
| LOC84664 | melanoma-associated chondroitin sulfate proteoglycan-like |
| RNASE1 | ribonuclease, RNase A family, 1 (pancreatic); RIB1; RNS1 |
| HAS1 | hyaluronan synthase 1; HAS |
| SLC16A8 | solute carrier 16 (monocarboxylic acid transporters), |
| | member 8; monocarboxylate transporter 3; MCT3 |
| CD164 | CD164 antigen, sialomucin; Sialomucin CD164; MGC-24; MUC-24 |
| FSTL1 | follistatin-like 1; follistatin-related protein; FRP; FSL1 |
| IL8 | interleukin 8; CXC chemokine ligand 8; |
| | LUCT/interleukin-8; T cell chemotactic factor; beta- |
| | thromboglobulin-like protein; emoctakin; granulocyte |
| | chemotactic protein 1; lymphocyte-derived neutrophil- |
| | activating factor; monocyte derived neutrophil-activating |
| | protein; monocyte-derived neutrophil chemotactic factor; |
| | neutrophil-activating factor; neutrophil-activating peptide |
| | 1; neutrophil-activating protein 1; protein 3-10C; small |
| | inducible cytokine subfamily B, member 8; 3-10C; |
| | AMCF-I; CXCL8; GCP-1; GCP1; IL-8; K60; LECT; |
| | LUCT; LYNAP; MDNCF; MONAP; NAF; NAP-1; |
| | NAP1; SCYB8; TSG-1; b-ENAP |
| KTN1 | kinectin 1 (kinesin receptor); CG-1 antigen; kinesin |
| | receptor; CG1; KIAA0004; KNT |
| RBP4 | retinol binding protein 4, plasma; retinol-binding protein 4, |
| | interstitial; retinol-binding protein 4, plasma |
| COL5A2 | collagen, type V, alpha 2; AB collagen; Collagen V, alpha- |
| | 2 polypeptide; collagen, fetal membrane, A polypeptide |
| TSPAN-3 | tetraspan TM4SF; tetraspanin 3; tetraspanin TM4-A; |
| | transmembrane 4 superfamily member 8 |
| CD63 | CD63 antigen (melanoma 1 antigen); granulophysin; |
| | lysosome-associated membrane glycoprotein 3; melanoma |
| | 1 antigen; melanoma-associated antigen ME491; |
| | melanoma-associated antigen MLA1; ocular melanoma- |
| | associated antigen; LAMP-3; ME491; MLA1; OMA81H |
| IGFBP3 | insulin-like growth factor binding protein 3; IBP3 |
| PLEC1 | plectin 1, intermediate filament binding protein, 500 kD; |
| | plectin 1, intermediate filament binding protein, 500 kD; EBS1; PCN; |
| | PLTN |
| CXCL2 | chemokine (C-X-C motif) ligand 2; GRO2 oncogene; |
| | CINC-2a; GRO2; GROB; GROb; MGSA-b; MIP-2a; |
| | MIP2; MIP2A; SCYB2 |
| GPR48 | G protein-coupled receptor 48; G-protein-coupled receptor 48; LGR4 |
| FLJ20559 | chromosome 9 open reading frame 95 (C9orf95), NRK1, |
| | FLJ20559, bA235O14.2 |
| LAMB1 | laminin, beta 1 |
| COL4A1 | collagen, type IV, alpha 1; collagen IV, alpha-1 |
| | polypeptide; collagen of basement membrane, alpha-1 |
| | chain |
| TFPI2 | tissue factor pathway inhibitor 2; PP5; TFPI-2 |
| ESR1 | estrogen receptor 1; estrogen receptor 1 (alpha); ER; ESR; ESRA; Era; |
| | NR3A1 |
| SLC11A3 | solute carrier family 11 (proton-coupled divalent metal ion |
| | transporters), member 3; iron regulated gene 1; |
| | FERROPORTIN 1; FPN1; Homo sapiens solute carrier |
| | family 11 (proton-coupled divalent metal ion transporters), |
| | member 3 (SLC11A3), mRNA.; IRON-REGULATED |
| | TRANSPORTER 1; IREG1; SOLUTE CARRIER |
| | FAMILY 11, MEMBER 3; SLC11A3; ferroportin 1; iron |
| | regulated gene 1; solute carrier family 11 (proton-coupled |
| | divalent metal ion transporters), member 3; FPN1; HFE4; |
| | IREG1; MTP1; NM_014585.1; SLC11A3 |
| EFNA1 | ephrin-A1; eph-related receptor tyrosine kinase ligand 1; |
| | eph-related receptor tyrosine kinase ligand 1 (tumor |
| | necrosis factor, alpha-induced protein 4); immediate early |
| | response protein B61; tumor necrosis factor, alpha-induced |
| | protein 4; B61; ECKLG; EFL1; EPLG1; LERK1; TNFAIP4 |
| KLK13 | kallikrein 13; kallikrein-like gene 4; DKFZP586J1923; KLK-L4; KLKL4 |
| AB065858 | seven transmembrane helix receptor |
| MMP7 | matrix metalloproteinase 7 (matrilysin, uterine); matrin; |
| | uterine matrilysin; MMP-7; MPSL1; PUMP-1 |
| INHBB | inhibin, beta B (activin AB beta polypeptide); Inhibin, |
| | beta-2; activin AB beta polypeptide precursor |
| PI3 | protease inhibitor 3, skin-derived (SKALP); WAP four- |
| | disulfide core domain 14; elafin precursor; elastase- |
| | specific inhibitor; skin-derived antileukoproteinase; |
| | ELAFIN; ESI; MGC13613; SKALP; WAP3; WFDC14 |

1. A bivalent molecule, comprising
a) two binding moieties, each binding moiety specifically binding to a compound produced by or associated with a the surface of a cancer cell, and wherein the each binding moiety binds to specifically binding to a different compound, and
b) a therapeutic agent or imaging agent.

2. The bivalent molecule of claim 1, wherein each binding moiety specifically binds to a compound selected from the group consisting of a compound of Table A, a compound of Table B, and a compound of Table C.

3. The bivalent molecule of claim 2, wherein one binding moiety specifically binds to a compound of Table A.

4. The bivalent molecule of claim 3, wherein the other binding moiety specifically binds to a compound of Table A.

5. The bivalent molecule of claim 3, wherein the other binding moiety specifically binds to a compound of Table B.

6. The bivalent molecule of claim 3, wherein the other binding moiety specifically binds to a compound of Table C.

7. The bivalent molecule of claim 2, wherein one binding moiety specifically binds to a compound of Table B.

8. The bivalent molecule of claim 7, wherein the other binding moiety specifically binds to a compound of Table B.

9. The bivalent molecule of claim 7, wherein the other binding moiety specifically binds to a compound of Table C.

10. The bivalent molecule of claim 2, wherein one binding moiety specifically binds to a compound of Table C.

11. The bivalent molecule of claim 10, wherein the other binding moiety specifically binds to a compound of Table C.

12. The bivalent molecule of claim 1, wherein the therapeutic agent is selected from the group consisting of a radioisotope, a drug or a toxin.

13. The method of claim 12, wherein the radioisotope is selected from the group consisting of 1-131,1-125, At-211, P-32, P-33, Sc-47, Cu-64, Cu-67, As-77, Y-90, Ph-05, Pd-109, Ag-111, Pr-143, Sm-153, Th-161, Ho-166, Lu-177, Re-186, Re-188, Re-189, Ir-194, Au-199, Pb-212, Bi-212.

14. The bivalent molecule of claim 1, wherein the imaging agent is selected from the group consisting of an imaging contrast agent, an ultrasound imaging agent, and a nuclear imaging agent.

15. The bivalent molecule of claim 14, wherein the imaging agent is selected from the group consisting of Tc-99m, In-111, Ga-67, Rh-105, I-123, Nd-147, Pm-151, Sm-153, Gd-159, Th-161, Er-171, Re-186, Re-188, and Tl-201.

16. A method of treating cancer, comprising administering the bivalent molecule of claim 1 to a patient in need thereof.

17. The method of claim 16, wherein the cancer is selected from the group consisting of carcinomas, melanomas, sarcomas, neuroblastomas, leukemias, lymphomas, gliomas, myelomas, breast cancers, colon cancers, lung cancers, renal cancers, ovarian cancers, prostate cancers, and uterine cancers.

18. A method of detecting cancer, comprising administering the bivalent molecule of claim 1 to a patient in need thereof.

19. The method of claim 18, wherein the cancer is selected from the group consisting of carcinomas, melanomas, sarcomas, neuroblastomas, leukemias, lymphomas, gliomas, myelomas, breast cancers, colon cancers, lung cancers, renal cancers, ovarian cancers, prostate cancers, and uterine cancers.
